(19) United States
(12) Patent Application Publication
(10) Pub. No.: US 2024/0287013 A1
(43) Pub. Date: Aug. 29, 2024

(54) A method for asymmetric synthesis of (-)- Anisomelic Acid

(71) Applicants: Gansu Evergreen Pharmaceuticals Co.,Ltd., Lanzhou City (CN); Peking University Shenzhen Graduate School, Shenzhen City (CN); CNS BIOTEK CORP., Taichung City (TW)

(72) Inventors: Zhen Yang, Shenzhen City (CN); Yew-Min Tzeng, Taichung City (TW); Hai-Xin Yu, Shenzhen City (CN); Nan Zheng, Shenzhen City (CN); Chong-Guo Jiang, Shenzhen City (CN)

(73) Assignees: Peking University Shenzhen Graduate School, Shenzhen City, GD (CN); CNS BIOTEK CORP., Taichung City, TW (TW); Gansu Evergreen Pharmaceuticals Co.,Ltd., Lanzhou City, GS (CN)

(21) Appl. No.: 18/565,537

(22) PCT Filed: Jun. 10, 2021
(86) PCT No.: PCT/CN2021/099492

§ 371 Date: May 17, 2024

(51) Int. Cl. C07D 307/93; C07D 307/58; C07F 9/40

(52) U.S. Cl. CPC C07D 307/93; C07D 307/58; C07F 9/4084

(57) ABSTRACT

A method for asymmetric synthesis of (−)-Anisomelic Acid is provided in the present invention, a chiral compound (−)-Costunolide is used as a starting material, a key intermediate is obtained by a regioselective ozone decomposition reaction, then carbon chain extension is performed by a Horner-Wadsworth-Emmons (HWE) reaction and a Peterson olefination reaction, and a (−)-anisomelic acid fourteen-membered carbocyclic skeleton is constructed by a ring-closing metathesis (RCM) reaction, laying an important foundation for subsequent (−)-anisomelic acid biological activity research, in the synthesis route, various (−)-anisomelic acid analogs can also be obtained from the key intermediate, the reaction operations in the synthesis route are simple and the present invention can be widely popularized and used.

### FIELD OF THE INVENTION

The present invention belongs to the field of organic chemical synthesis and relates to a method for asymmetric synthesis of (−)-Anisomelic Acid completed by a synthetic strategy expanding 14-membered macrocycle from 10-membered cycle through ozone decomposition, Horner-Wadsworth-Emmons (HWE) reaction, Peterson alkylene reaction, and ring-closing metathesis (RCM) reaction.

### BACKGROUND OF THE INVENTION

The totally synthetic (±)-anisomelic acid racemic mixture was first seen in 1987. In the last decades, a research team of Abo Akademi University which is the only Swedish-language teaching university in Finland has conducted a series of studies on the protection of anisomelic acid and its derivatives against cervical cancer caused by human papillomavirus.

In the past 20 years, the inventor's team has been carrying out a long-term breeding of “Hakka wipe grass” namely Anisomeles indica O. Kuntze (GenBank: GU726292) and has continuously conduct a series of research on the whole grass extract of the Anisomeles indica O. Kuntze planted in Zixiu Farm, Yuli Town, Hualien County, Taiwan. Specifically, the extraction, separation, purification, analysis, identification of the natural Anisomeles indica O. Kuntze, and the pharmacologic effect research such as anti-inflammation, anti-fatigue, anti-allergy, anti-asthma, anti-influenza virus, anti-Helicobacter pylori, anti-cancer, anti-cancer stem cells, etc. were carried out. In particular, the three-dimensional structure of the crystalline pure substance of the natural substance (−)-anisomelic acid contained in Anisomeles indica O. Kuntze was confirmed. The chemical formula (−)-anisomelic acid of the inventive feature is shown in FIG. 1.

In summary, (−)-anisomelic acid is a valuable molecular probe that can be used to study the mechanism of anticancer biological activity.

The natural substance (−)-anisomelic acid is a natural diterpenoid compound that is extracted from Anisomeles indica O. Kuntze, and the content of the (−)-anisomelic acid in the whole plant of Anisomeles indica O. Kuntze is generally about 70 to 100 ppm of dry weight. Obviously, (−)-anisomelic acid has low abundance in nature, difficulty in extraction, limited source, and lack of (−)-anisomelic acid and derivatives thereof hindering the comprehensive biological study of anticancer. At present, total synthesis of (−)-anisomelic acid has not been reported.

### DETAILED DESCRIPTION OF THE INVENTION

In order to promote the comprehensive biological study of (−)-Anisomelic Acid against cancer, the present invention provides a method for asymmetric synthesis of (−)-Anisomelic Acid completed by a synthetic strategy expanding 14-membered macrocycle from 10-membered cycle through ozone decomposition, HWE reaction, Peterson alkylene reaction, and RCM reaction. The reaction in the synthesis is simple to be operated and can be widely popularized and used, which provides sufficient samples for its activity testing, and lays a foundation for further structural optimization of complex macrocyclic skeleton small molecules and the development of highly active and highly selective anticancer drugs.

The chemical structure of (−)-Anisomelic Acid is shown below:

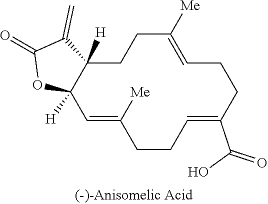 [SMILES: C=C1C(=O)O[C@@H]2/C=C(\C)CC/C=C(\C(=O)O)CC/C=C(\C)CC[C@@H]12]

In order to achieve the above purpose, the present invention adopts the following technical solution:

A method for asymmetric synthesis of (−)-Anisomelic Acid, comprising the following steps:
1) preparing an aldehydes and ketone compound 1 by using a chiral compound (−)-Costunolide as starting material under the condition of ozonative decomposition;
2) preparing unsaturated lactone compound (Z)-3 and unsaturated lactone compound (E)-3 by using the aldehydes and ketone compound 1 and a compound 6 under alkaline condition;
3) preparing tetraene compound 4 by using the unsaturated lactone compound (Z)-3 under 1,2-addition condition promoted by cerium trichloride and the subsequent elimination;
4) preparing a 14-membered macrocyclic compound (Z)-5 and a 14-membered macrocyclic compound (E)-5 by using the tetraene compound 4 under the condition of olefin metathesis; and
5) preparing a natural product (−)-Anisomelic Acid by using the 14-membered macrocyclic compound (E)-5 under the conditions of silica removal and hydrolysis.

The chemical formulae of each compound are shown in FIG. 2. The R group in the compound 6, the compound (Z)-3, the compound (E)-3, the compound 4, the compound (E)-5 and the compound (Z)-5 can be alkoxy group, aromatic oxygen group, alkylamine group, aromatic amine group, alkyl sulfhydryl group, aromatic sulfhydryl group, silicon group.

Further, the method of step 1) that preparing an aldehydes and ketone compound 1 under the condition of ozonative decomposition by using a chiral compound (−)-Costunolide as starting material comprises:

Ozone is introduced into the compound (−)-Costunolide in solution at −78° C., the reaction is monitored by thin-layer chromatography, the reducing reagent dimethyl sulfide is added to quench the reaction after the end of the reaction. After raising the reaction system to room temperature, removing solvents, and purifying the residue by using silica gel column chromatography to obtain compound 1.

The solvent in the reaction is selected to be mixed solvent, dichloromethane-methanol, dichloromethane-acetone, dichloromethane-acetic acid, all of which can obtain compound 1. The reducing quenching reagent can be dimethyl sulfide, triphenylphosphine. If acetic acid is used as a co-solvent, in addition to the addition of reducing quenching reagent, it is necessary to neutralize the acetic acid in the reaction system with a saturated sodium bicarbonate solution. Sudan III can also be used in the reaction as an indicator to monitor whether the reaction is complete.

Further, the method of step 2) that preparing unsaturated lactone compound (Z)-3 and unsaturated lactone compound (E)-3 by using the aldehydes and ketone compound 1 and a phosphate compound 6 under alkaline condition comprises:

At −78° C., alkaline substance is added dropwise into the tetrahydrofuran solution of compound 6, and after stirring at this temperature for 30 minutes, the tetrahydrofuran solution of compound 1 is added, and the quencher is added after the end of the reaction, and the residue is purified by using silica gel column chromatography to obtain compound (Z)-3 and compound (E)-3.

The unsaturated lactones are α, β-unsaturated lactones, the alkaline substances can be selected to be sodium hexamethylsilylamide, potassium hexamethylsilylamide, lithium hexamethylsilylamide, large steric hindrance alkaline substances which are not easy to occur Michael reaction to exocyclic double bonds. The choices of the solvents, the reagents, and the alkaline substances for the reaction can influence the ratios of compound (Z)-3 and compound (E)-3.

Further, the method of step 3) that preparing tetraene compound 4 by using the unsaturated lactone compound (Z)-3 under 1,2-addition condition promoted by cerium trichloride and the subsequent elimination comprises:

Adding the cerium trichloride into a round-bottom bottle, heating to 135˜150° C. under vacuum condition, and stirring for a certain time (such as 3 hours), filling with inert gas, moving the reaction system into an ice water bath, adding tetrahydrofuran, and then raising the temperature to room temperature and stirring for a certain time (for example: 12 hours). Reducing the temperature of the above reaction system to −78˜−80° C., adding n-pentane solution of (Trimethylsilyl)methyllithium reagent dropwise, and keeping the same temperature and continuously stirring for a certain time (such as 1.5 hours), then, adding compound (Z)-3 into the above reaction system, and stirring for a certain time (such as 1.5 hours) under the condition of −78˜−80° C. Quenching the reaction system by adding acetic acid aqueous solution, separating the liquid, and extracting the aqueous phase by ethyl acetate. Combining the organic phase, drying, removing the solvent, redissolving the residue in dichloromethane after the residue is spun dry, adding silica gel for promoting elimination, spinning dry the solvent after stirring for 24 hours, separating the residue by silica gel column chromatography to obtain compound 4.

The quality of cerium trichloride has an extremely important effect on the reaction. The reagent for promoting elimination can be acidic substances or alkaline substances, such as concentrated sulfuric acid, potassium tert-butoxide.

Further, the method of step 4) that preparing a 14-membered macrocyclic compound (Z)-5 and a 14-membered macrocyclic compound (E)-5 under the condition of olefin metathesis by using the tetraene compound 4 is:

After adding catalyst of olefin metathesis into the tetraene compound 4 in solution, discharging the residual oxygen from the reaction system under the condition of inert gas atmosphere for a certain time. Subsequently, heating the reaction system to 60° C. until the conversion of tetraene compound 4 being complete. Removing the solvents, purifying the residue by using silica gel column chromatography to obtain the 14-membered macrocyclic compound (Z)-5 and the 14-membered macrocyclic compound (E)-5.

The reaction solvent, reaction concentration, reaction temperature, and the selection of reaction catalyst have an important impact on the compounds generated by the reaction and the reaction time.

Further, the method of step 5) that preparing a natural product (−)-Anisomelic Acid under the conditions of silica removal and hydrolysis by using the 14-membered macrocyclic compound (E)-5 comprises:

Cooling down the tetrahydrofuran solution of the 14-membered macrocyclic compound (E)-5 to 0° C., adding desiliconization reagent tetrabutylammonium fluoride solution dropwise and reacting at the temperature for 1 hour, quenching the reaction system by using saturated ammonium chloride solution, extracting by using ethyl acetate and combining the organic phase after raising the temperature to room temperature; Drying and removing solvent, purifying the residue by using silica gel column chromatography to obtain the natural product (−)-Anisomelic Acid.

The desiliconization reagent can choose tetrabutylammonium fluoride, hydrofluoric acid aqueous solution, etc.

The step 2) further preparing a key intermediate compound 6 through nucleophilic substitution reaction, comprising the following steps:

2-1) preparing phosphate compound 6 by using compound 2 and compound 3 under alkaline condition;

The chemical formulae of compound 2 and compound 3 are shown in FIG. 3. In compound 2, the R₁ group can be alkoxy group, aromatic oxygen group, alkylamine group, aromatic amine group, alkyl sulfhydryl group, aromatic sulfhydryl group, silicon group. R₃ group can be phenyl group, trifluoroethyl group.

In compound 3, R₂ group can be chlorine, bromine, iodine, methylsulfonyloxy, p-toluene sulfonyloxy, trifluoromethylsulfonyloxy.

Further, the method of step 2-1) that preparing phosphate compound 6 under alkaline condition by using compound 2 and compound 3 comprises:

Cooling down the tetrahydrofuran solution of compound 2 to 0° C., adding alkaline substance sodium hydride slowly under an inert gas atmosphere. Stirring for a certain time at the temperature, slowly adding the tetrahydrofuran solution of compound 3 in a dropwise manner, and then raising the temperature until the reaction being complete, quenching the reaction system by using saturated ammonium chloride solution, after the temperature being increased to room temperature, extracting by using ethyl acetate, and combining the organic phase; Drying, removing solvent, purifying the residue by using silica gel column chromatography to obtain compound 6.

The R₂ group in compound 3 has a great influence on the reaction time.

The above reactions that need to be carried out under an inert gas atmosphere are preferably carried out in an argon gas atmosphere.

The extraction of the reactions is preferably completed by using ethyl acetate.

The dry of the above steps is preferably to dry the organic phase with anhydrous sodium sulfate, and the solvent removal is to remove the solvent by using a rotary evaporator.

Preferably, in step 1), dichloromethane-acetic acid is used as the mixed solvent, acetic acid can react with secondary ozone oxides, and the generated peroxide intermediates are much easier reduced to compound 1. The reagent for reductive quenching is preferably chosen dimethyl sulfide, and the products are easier to be separated and purified after the reaction.

Preferably, in step 2), the alkaline reagent is sodium hexamethylsilylamide, the solvent is tetrahydrofuran, and the obtained compound (Z)-3 ratio is the highest.

Preferably, in step 3), the cerium trichloride is anhydrous cerium trichloride, it is also available if cerium trichloride with crystalline water is chosen and needs to be ground into powder, but it requires a better drying process and a longer drying time.

Choosing weakly acidic silica gel as the elimination accelerator can make the yield of compound 4 be the highest.

Preferably, in step 4), since the catalyst of Hoveyda-Grubbs II has a better thermal stability, the using amount of the catalyst can be reduced. The reaction concentration is controlled at about 0.005M, which can effectively avoid the formation of intermolecular olefin metathesis products.

Preferably, in step 5), the tetrahydrofuran solution of anhydrous tetrabutylammonium fluoride is able to provide the highest yields.

Preferably, in step 2-1), the R₂ group of compound 3 is iodine, which can reduce the reaction time without the necessary of adding iodide as an accelerator.

In the present invention, the compounds 2 and 3 are known compounds, that is, the compounds 2 and 3 may not be prepared by the method of the present invention, but by adopting the existing compound products, and the other compounds must be prepared by the method of the present invention.

The technical effects of the present invention are as follows:

The above mentioned asymmetric synthesis of (−)-Anisomelic Acid is achieved by ozonation decomposition of the chiral compound (−)-Costunolide, followed by the extension of the carbon chain, and the construction of a 14-membered macrocyclic skeleton by the synthesis strategy of RCM reaction, that is, the basis of the present invention is to prepare (−)-Anisomelic acid from (−)-Costunolide, as shown in FIG. 4.

The present invention develops a regioselective ozonative decomposition to cut off the double bond from the chiral compound (−)-Costunolide with 10-membered carbon ring, and then completes the extension of the carbon chain by HWE reaction and Peterson alkylene, and obtains the key 14-membered carbon ring skeleton structure of (−)-Anisomelic acid through RCM reaction, and then completes the total synthesis of (−)-Anisomelic acid by removing the silicon group.

### BRIEF DESCRIPTION OF THE DRAWINGS

FIG. 1 illustrates the chemical formula of (−)-Anisomelic acid of the present invention.

FIG. 2 illustrates each compound used or produced in the asymmetric synthesis of the present invention.

FIG. 3 shows the chemical formulae of compound 2 and compound 3.

FIG. 4 shows the analysis process of retrosynthesis for preparing (−)-Anisomelic acid from (−)-Costunolide of the present invention.

FIG. 5 shows the synthesis of compound 1.

FIG. 6 shows the synthesis of compound 6.

FIG. 7 shows the synthesis of compound (Z)-3 and compound (E)-3.

FIG. 8 shows the synthesis of compound 4.

FIG. 9 shows the synthesis of compound (Z)-5 and compound (E)-5.

FIG. 10 shows the synthesis of natural product (−)-Anisomelic acid.

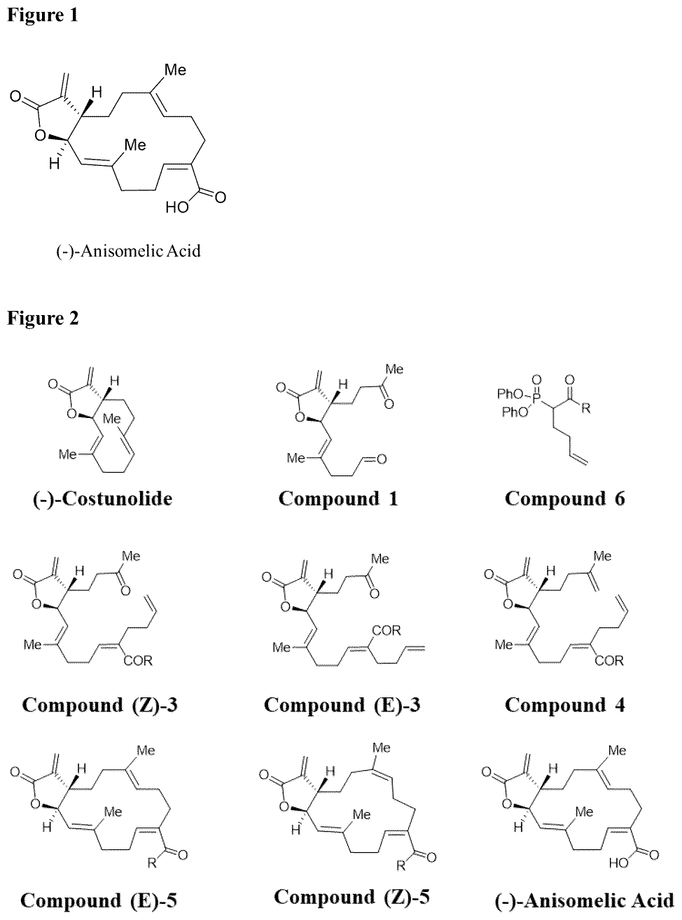

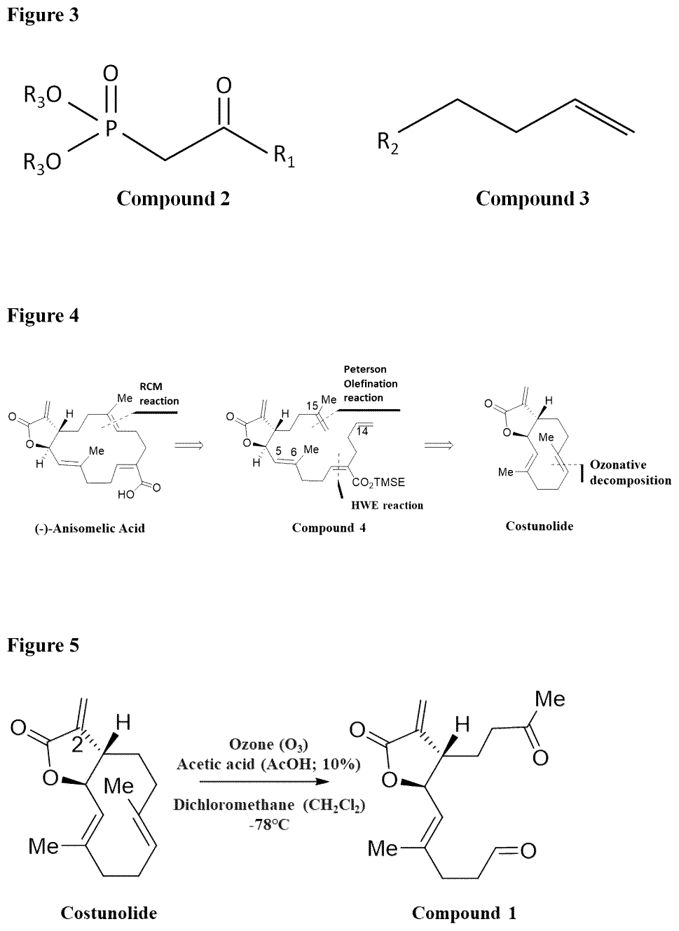

### DESCRIPTION OF EMBODIMENTS

The technical solution of the present invention is further illustrated below through specific examples, and the specific examples do not represent any limitation on the scope of protection of the present invention. Some non-essential modifications and adjustments made by others according to the concept of the present invention still belong to the scope of protection of the present invention.

### Example 1. Synthesis of Compound 1

As shown in FIG. 5. (−)-Costunolide (800 mg, 3.54 mmol) was dissolved in dichloromethane (250 mL) containing acetic acid (25 mL, 10% v/v), and the resulting mixture was cooled down to −78° C., ozone was carefully introduced into the reaction system, and the reaction process was monitored by thin-layer chromatography until (−)-Costunolide was completely consumed. Dimethyl sulfide (1.0 mL) was added, and the mixture temperature was slowly raised to room temperature, and the saturated sodium bicarbonate solution (200 mL) was slowly added into the reaction system, and then the mixture was extracted with ethyl acetate (3*200 mL). The combined organic phase was washed by saturated saline (300 mL) and dried by sodium sulfate. The solvent was removed by vacuum and the residue was purified by silica gel column chromatography (petroleum ether: ethyl acetate=8:1 to 4:1) to obtain a colorless oily compound 1 (773 mg, 85% yield).

The assay data for compound 1 were as follows:

R_{f}=0.25 (ethyl acetate/petroleum ether=1/1).

[α]_{D}²²=+26.1 (c=0.12, CHCl₃).

¹H NMR (400 MHZ, CDCl₃) δ 9.75 (s, 1H), 6.24 (d, J=2.8 Hz, 1H), 5.57 (d, J=2.5 Hz, 1H), 5.17 (d, J=8.3 Hz, 1H), 4.75 (dd, J=8.9, 6.1 Hz, 1H), 2.72 (dt, J=8.3, 5.8 Hz, 1H), 2.57-2.37 (m, 5H), 2.19-2.06 (m, 4H), 1.95 (dt, J=13.7, 7.4 Hz, 1H), 1.85-1.75 (m, 4H).

¹³C NMR (101 MHz, CDCl₃) δ 207.29, 201.26, 170.00, 142.50, 138.76, 123.25, 122.02, 79.36, 45.06, 41.53, 39.69, 31.39, 30.07, 25.66, 17.17.

IR vₘₐₓ (film): 2949, 2730, 1726, 1684, 1450, 1389, 1250, 1189, 737 cm⁻¹.

HRMS (ESI) m/z: C₁₅H₂₀NaO₄ [M+Na]+: calculated value: 287.1254; measured value: 287.1248.

### Example 2. Synthesis of Compound 6

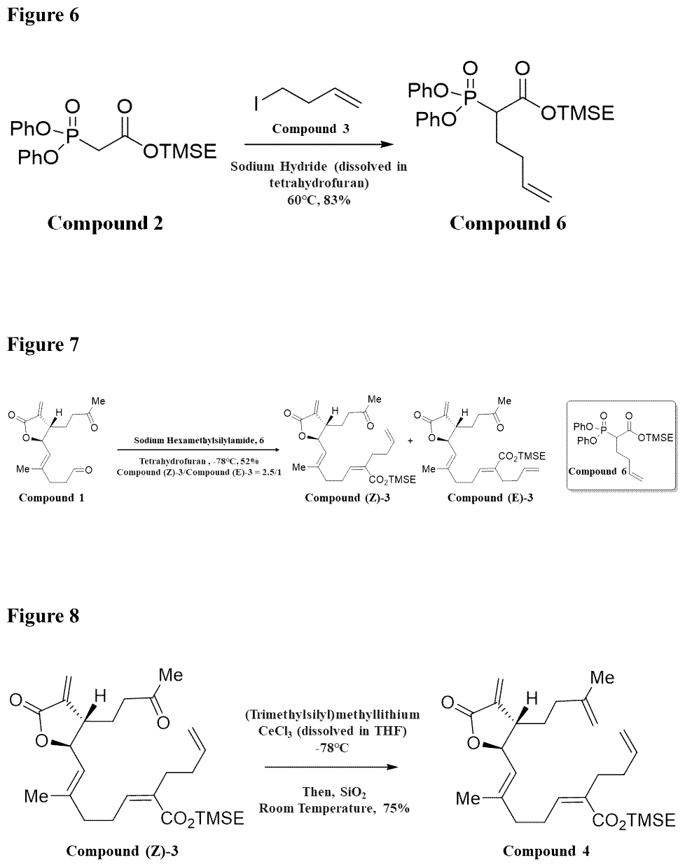

As shown in FIG. 6. Compound 2 (5.0 g, 12.7 mmol) was dissolved in tetrahydrofuran (350 mL), sodium hydride (60% dispersion in mineral oil, 720 mg) was added into the stirring solution in batches at 0° C., bubbles were generated, the mixture was re-stirred at room temperature for 1 hour, the tetrahydrofuran (10 mL) solution of compound 3 (3.9 g, 21.6 mmol) was slowly added dropwise into the reaction system, and then the mixture was heated to 60° C. for 48 hours. After the monitoring was completed by thin-layer chromatography, saturated ammonium chloride solution (200 mL) was slowly added into the reaction system to quench the reaction, and then the mixture was extracted with ethyl acetate (3*150 mL). The combined organic extracts were washed by saturated salt water (500 mL) and dried by sodium sulfate. The solvent was vacuum concentrated, and the residue was purified by silica gel column chromatography (petroleum ether:ethyl acetate=20:1), and a colorless oily compound 6 was obtained, (4.7 g, 83% yield).

The assay data for compound 6 were as follows:

R_{f}=0.5 (ethyl acetate/petroleum ether=1/10).

¹H NMR (500 MHz, CDCl₃) δ 7.31 (dd, J=14.9, 7.4 Hz, 4H), 7.18 (dd, J=13.6, 6.4 Hz, 6H), 5.77 (ddt, J=12.6, 10.2, 6.2 Hz, 1H), 5.06 (dd, J=13.7, 7.1 Hz, 2H), 4.31-4.21 (m, 2H), 3.31 (ddd, J=23.1, 10.5, 2.8 Hz, 1H), 2.39-2.08 (m, 4H), 1.06-0.95 (m, 2H), 0.04 (d, J=0.6 Hz, 9H).

¹³C NMR (126 MHz, CDCl₃) δ 168.34, 136.49, 129.85, 125.46, 120.66, 116.68, 115.50, 64.38, 45.88, 44.82, 32.34, 32.21, 26.27, 26.23, 17.50, −1.45.

IR vₘₐₓ (film): 3442, 2920, 1696, 1415, 1257, 1230, 861 cm⁻¹.

HRMS (ESI) m/z: C₂₃H₃₁NaO₅PSi [M+Na]+: calculated value: 496.1571; measured value: 496.1571.

### Example 3. Compound (Z)-3 and Compound (E)-3

As shown in FIG. 7. Compound 6 (1.0 g, 2.27 mmol) was dissolved in tetrahydrofuran (100 mL), sodium hexamethylsilylamide solution (1.0 mL, 2.0 M THE solution) was slowly added dropwise into the reaction system at −78° C., after the dropwise addition was completed, continuously stirred at −78° C. for 1 hour, and then compound 1 (500 mg, 1.89 mmol) dissolved in THF (20 mL) was added dropwise, after 30 minutes of reaction, the reaction process was monitored by thin-layer chromatography. Saturated ammonium chloride solution (100 mL) was added into the reaction system to quench the reaction, and then the mixture was extracted with ethyl acetate (3*150 mL). The combined organic phase was washed by saturated saline (500 mL) and dried by sodium sulfate. The solvent was vacuum concentrated, and the residue was purified by silica gel column chromatography (petroleum ether:ethyl acetate=8:1), and the colorless oily compound (Z)-3 (514 mg, 59% yield) and colorless oily compound (E)-3 (201 mg, 23% yield) were obtained.

The assay data for compound (Z)-3 were as follows:

R_{f}=0.4 (ethyl acetate/petroleum ether=1/1).

[α]_{D}²¹=+24.0 (c=0.1, CHCl₃).

¹H NMR (500 MHZ, CDCl₃) δ 6.28 (d, J=2.9 Hz, 1H), 5.82-5.72 (m, 2H), 5.58 (d, J=2.5 Hz, 1H), 5.20 (dd, J=9.1, 1.2 Hz, 1H), 5.03-4.93 (m, 2H), 4.80 (dd, J=9.1, 5.9 Hz, 1H), 4.25-4.19 (m, 2H), 2.78-2.71 (m, 1H), 2.58 (dd, J=15.2, 7.4 Hz, 2H), 2.50 (t, J=7.6 Hz, 2H), 2.35-2.30 (m, 2H), 2.20-2.13 (m, 7H), 1.98 (ddd, J=14.0, 7.4, 6.0 Hz, 1H), 1.84 (td, J=14.4, 7.7 Hz, 1H), 1.78 (d, J=1.3 Hz, 3H), 1.06-1.01 (m, 2H), 0.05 (s, 9H).

¹³C NMR (126 MHz, CDCl₃) δ 207.06, 167.97, 143.67, 140.51, 138.92, 137.80, 132.35, 122.89, 121.85, 115.09, 79.54, 62.46, 45.14, 39.66, 39.06, 34.00, 33.36, 30.08, 27.43, 25.76, 17.53, 16.99, −1.47 ppm.

IR vₘₐₓ (film): 3310. 2926, 2375, 1507, 1262, 1019, 1011, 851, 837, 799 cm⁻¹.

HRMS (ESI) m/z: C₂₆H₄₀NaO₅Si [M+Na]+: calculated value: 483.2537; measured value: 483.2537.

The assay data for compound (E)-3 were as follows:

R_{f}=0.35 (ethyl acetate/petroleum ether=1/1).

[α]_{D}²²=+31.8 (c=0.17, CHCl₃).

¹H NMR (500 MHZ, CDCl₃) δ 6.70 (t, J=7.3 Hz, 1H), 6.29 (d, J=2.9 Hz, 1H), 5.80 (ddt, J=17.0, 10.1, 6.8 Hz, 1H), 5.59 (d, J=2.5 Hz, 1H), 5.22 (dd, J=9.0, 1.2 Hz, 1H), 5.05-4.93 (m, 2H), 4.80 (dd, J=9.0, 5.9 Hz, 1H), 4.26-4.18 (m, 2H), 2.83-2.69 (m, 1H), 2.57-2.43 (m, 2H), 2.42-2.35 (m, 2H), 2.31 (dd, J=15.2, 7.5 Hz, 2H), 2.21-2.12 (m, 7H), 2.03-1.92 (m, 1H), 1.86 (tt, J=14.4, 7.2 Hz, 1H), 1.80 (d, J=1.2 Hz, 3H), 1.02 (ddd, J=10.5, 7.2, 3.8 Hz, 2H), 0.05 (s, 9H).

¹³C NMR (126 MHZ, CDCl₃) δ 207.06, 169.98, 167.78, 143.20, 141.06, 138.82, 137.91, 132.59, 123.21, 121.97, 115.12, 79.41, 62.76, 45.13, 39.67, 38.47, 33.37, 30.06, 26.66, 26.46, 25.77, 17.42, 17.06, −1.44.

IR vₘₐₓ (film): 3440, 3310, 2926, 2375, 1262, 1250, 1019, 1011, 861, 837, 799 cm⁻¹.

HRMS (ESI) m/z: C₂₆H₄₀NaO₅Si [M+Na]+: calculated value: 483.2537; measured value: 483.2539.

### Example 4. Synthesis of Compound 4

As shown in FIG. 8. Anhydrous cerium chloride (493 mg, 2.0 mmol) was added into a round-bottom flask, heated to 150° C. under vacuum conditions and stirred for 3 hours, filled with argon gas, moved the system to a 0° C. ice bath, tetrahydrofuran (5 mL) was added, and then raised to room temperature and stirred for more than 24 hours. The above system was cooled down to −78° C., (Trimethylsilyl)methyllithium (1.5 mL, 1.5 mmol, 1.0 M n-pentane solution) was added dropwise, the same temperature was maintained and stirred for 1 hour, and then compound (Z)-3 (460 mg, 1.0 mmol, in 2.0 mL tetrahydrofuran) was added into the above system and stirred at −78° C. for 1 hour. After the detection reaction was completed by thin-layer chromatography, 10% acetic acid aqueous solution (10 mL) was added to quench, the liquid was separated, and the aqueous phase was extracted with dichloromethane, the organic phase was combined, dried, and the solvent was removed under reduced pressure. The residual silica gel was separated by column chromatography (petroleum ether/ethyl acetate=20:1) to obtain colorless oily compound 4 (341 mg, 75% yield).

The assay data for compound 4 were as follows:

R_{f}=0.5 (ethyl acetate/petroleum ether=1/10).

[α]_{D}²³=+32.7 (c=0.35, CHCl₃).

¹H NMR (500 MHZ, CDCl₃) δ 6.26 (d, J=2.8 Hz, 1H), 5.82-5.70 (m, 2H), 5.57 (d, J=2.5 Hz, 1H), 5.22 (dd, J=9.1, 0.8 Hz, 1H), 5.04-4.92 (m, 2H), 4.84 (dd, J=9.1, 5.7 Hz, 1H), 4.76 (s, 1H), 4.68 (s, 1H), 4.25-4.19 (m, 2H), 2.74-2.67 (m, 1H), 2.57 (dd, J=15.1, 7.4 Hz, 2H), 2.34-2.27 (m, 2H), 2.19-2.12 (m, 4H), 2.05 (t, J=7.9 Hz, 2H), 1.86-1.75 (m, 4H), 1.75-1.62 (m, 4H), 1.10-0.96 (m, 2H), 0.05 (s, 9H).

¹³C NMR (126 MHz, CDCl₃) δ 170.31, 167.99, 144.37, 142.94, 140.58, 139.42, 137.81, 132.27, 123.19, 121.50, 115.06, 110.96, 79.85, 62.44, 45.48, 39.05, 34.37, 34.02, 33.38, 30.77, 27.36, 22.41, 17.52, 16.92, −1.47.

IR vₘₐₓ (film): 2845, 2410, 1825, 1260, 1176, 1132, 1114, 1012, 934, 857, 835, 797 cm⁻¹.

HRMS (ESI) m/z: C₂₇H₄₂NaO₄Si [M+Na]+: calculated value: 481.2745; measured value: 481.2743.

### Example 5. Synthesis of Compound (Z)-5 and Compound (E)-5

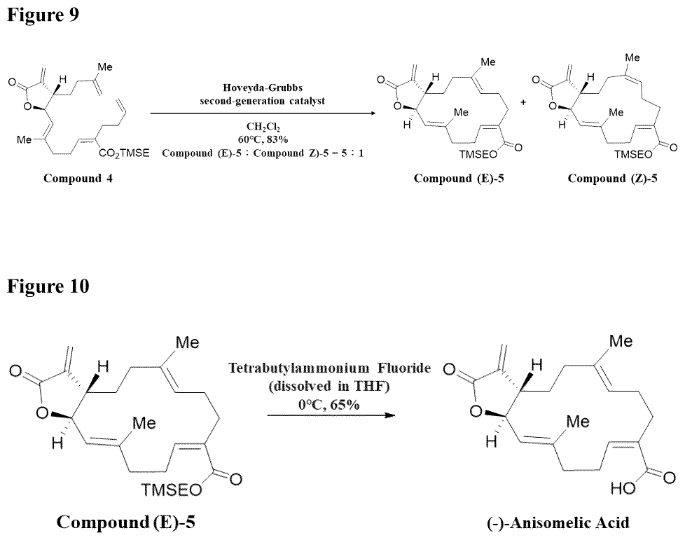

As shown in FIG. 9. The compound 4 (100 mg, 0.22 mmol) was dissolved in dichloromethane (1.0 L), Hoveyda-Grubbs second-generation catalyst (6.8 mg, 0.01 mmol) was added at room temperature. Subsequently, argon gas was introduced into the reaction system for 30 minutes. After the gas was replaced in the reaction system, the temperature was raised to 60° C. and stirred for 48 hours. After the thin-layer chromatography detection reaction was completed, the solvent was removed directly under reduced pressure. The residues were separated by silica gel column chromatography (petroleum ether/ethyl acetate=10:1) to obtain colorless oily compound (E)-5 (65 mg, 69% yield) and colorless oily compound (Z)-5 (13 mg, 14% yield).

The assay data for compound (E)-5 were as follows:

R_{f}=0.35 (ethyl acetate/petroleum ether=1/10).

[α]_{D}²⁴=−48.7 (c=0.24, CHCl₃).

¹H NMR (400 MHZ, CDCl₃) δ 6.24 (d, J=2.6 Hz, 1H), 5.64 (t, J=6.6 Hz, 1H), 5.57 (d, J=2.3 Hz, 1H), 5.21-5.15 (m, 1H), 5.05-4.96 (m, 1H), 4.88 (dd, J=9.7, 4.0 Hz, 1H), 4.23 (ddd, J=8.0, 5.0, 1.3 Hz, 2H), 2.86-2.73 (m, 1H), 2.71-2.56 (m, 2H), 2.47 (dd, J=13.1, 6.6 Hz, 1H), 2.32-2.02 (m, 7H), 1.78 (d, J=1.0 Hz, 3H), 1.76-1.67 (m, 2H), 1.59 (s, 3H), 1.07-0.99 (m, 2H), 0.05 (s, 9H).

¹³C NMR (101 MHZ, CDCl₃) δ 170.58, 168.13, 142.21, 141.22, 140.76, 132.19, 131.00, 125.61, 124.18, 121.66, 79.17, 62.52, 43.04, 38.49, 36.18, 34.71, 32.30, 25.71, 25.13, 17.67, 16.59, 15.76, −1.40.

HRMS (ESI) m/z: C₂₅H₃₈NaO₄Si [M+Na]+: calculated value: 453.2432; measured value: 453.2430.

The assay data for compound (Z)-5 were as follows:

R_{f}=0.25 (ethyl acetate/petroleum ether=1/10).

[α]_{D}²⁴=−12.1 (c=0.1, CHCl₃)

¹H NMR (400 MHZ, CDCl₃) δ 6.23 (d, J=3.2 Hz, 1H), 5.75 (dd, J=10.1, 4.2 Hz, 1H), 5.55 (d, J=2.9 Hz, 1H), 5.30 (d, J=8.9 Hz, 1H), 5.21 (t, J=7.9 Hz, 1H), 4.73 (t, J=8.6 Hz, 1H), 4.22-4.15 (m, 2H), 3.14-2.99 (m, 1H), 2.69 (dd, J=8.1, 3.3 Hz, 1H), 2.59-2.50 (m, 1H), 2.38-2.19 (m, 5H), 2.13-2.03 (m, 3H), 1.98-1.92 (m, 1H), 1.81 (s, 4H), 1.68 (s, 3H), 1.03 (dd, J=9.9, 7.6 Hz, 2H), 0.06 (s, 9H).

¹³C NMR (101 MHZ, CDCl₃) δ 170.50, 168.19, 145.53, 141.93, 140.27, 135.90, 134.14, 125.10, 123.71, 120.34, 80.01, 62.49, 47.17, 39.17, 35.26, 30.28, 29.93, 29.71, 25.84, 23.00, 17.73, 16.43, −1.38.

HRMS (ESI) m/z: C₂₅H₃₈NaO₄Si [M+Na]+: calculated value: 453.2432; measured value: 453.2430.

### Example 6. Synthesis of Natural Product (−)-Anisomelic Acid

As shown in FIG. 10. Compound (E)-5 (50 mg, 0.12 mmol) was dissolved in tetrahydrofuran (5 mL), tetrabutylammonium fluoride solution (0.17 mL, 0.17 mmol, 1.0 M tetrahydrofuran solution) was added at 0° C., stirred for 1 hour, the thin-layer chromatography detection reaction was completed, saturated ammonium chloride solution (10 mL) was added into the reaction system to quench the reaction, and then the mixture was extracted with ethyl acetate (3*5 mL). The combined organic phase was washed by saturated saline (10 mL) and dried by sodium sulfate. The solvent was vacuum concentrated, and the residue was purified by silica gel column chromatography (petroleum ether:ethyl acetate=4:1).

The assay data for natural product (−)-Anisomelic acid were as follows:

R_{f}=0.3 (ethyl acetate/petroleum ether=1/2).

[α]_{D}²⁴=22.8 (c=1.2, CHCl₃).

¹H NMR (500 MHZ, CDCl₃) δ 6.25 (d, J=2.6 Hz, 1H), 5.88 (t, J=6.5 Hz, 1H), 5.59 (d, J=2.3 Hz, 1H), 5.18 (d, J=9.6 Hz, 1H), 4.99 (d, J=5.3 Hz, 1H), 4.88 (dd, J=9.6, 4.2 Hz, 1H), 2.88 (ddd, J=21.6, 14.2, 7.1 Hz, 1H), 2.77-2.64 (m, 2H), 2.50 (t, J=13.4 Hz, 1H), 2.36-2.16 (m, 6H), 2.11-2.02 (m, 1H), 1.78 (d, J=0.8 Hz, 1H), 1.76-1.63 (m, 2H), 1.60 (s, 3H).

¹³C NMR (126 MHz, CDCl₃) δ 173.11, 170.62, 146.93, 141.15, 140.66, 132.52, 129.68, 125.36, 124.36, 121.77, 79.16, 43.07, 38.46, 36.18, 34.44, 32.21, 26.16, 25.08, 16.64, 15.83.

HRMS (ESI) m/z: C₂₀H₂₆NaO₄ [M+Na]+: calculated value: 353.1723; measured value: 353.1723.

The technical features of the above embodiments may be combined in any appropriate way, and to make the description concise, not all possible combinations of the individual technical features in the above embodiments are described. However, as long as there is no contradiction between the combinations of these technical features, they shall be considered to be within the scope of this specification.

The above embodiments only describe several embodiments of the present invention, which facilitates a specific and detailed understanding of the technical solution of the present invention, but cannot be construed as a restriction on the scope of protection of the invention. It should be noted that, for a person skilled in the art, under the premise of not departing from the conception of the present invention, a number of deformations and improvements can also be made, which are within the scope of protection of the present invention. It should be understood that on the basis of the technical solution provided in the present invention, the technical solution obtained by a person skilled in the art through logical analysis, reasoning or limited test is within the scope of protection of the claims described in the present invention.

What is claimed is:

1. A method for asymmetric synthesis of (−)-Anisomelic Acid, characterized by comprising:
1) preparing an aldehydes and ketone compound 1 by using a chiral compound (−)-Costunolide as starting material under the condition of ozonative decomposition;
2) preparing an unsaturated lactone compound (Z)-3 and an unsaturated lactone compound (E)-3 by using the aldehydes and ketone compound 1 and a phosphate compound 6 under alkaline condition;
3) preparing a tetraene compound 4 by using the unsaturated lactone compound (Z)-3 under 1,2-addition condition promoted by cerium trichloride and the subsequent elimination;
4) preparing a 14-membered macrocyclic compound (Z)-5 and a 14-membered macrocyclic compound (E)-5 by using the tetraene compound 4 under the condition of olefin metathesis; and
5) preparing a natural product (−)-Anisomelic Acid by using the 14-membered macrocyclic compound (E)-5 under the conditions of silica removal and hydrolysis;
wherein the chemical formulae of each compound are shown below: 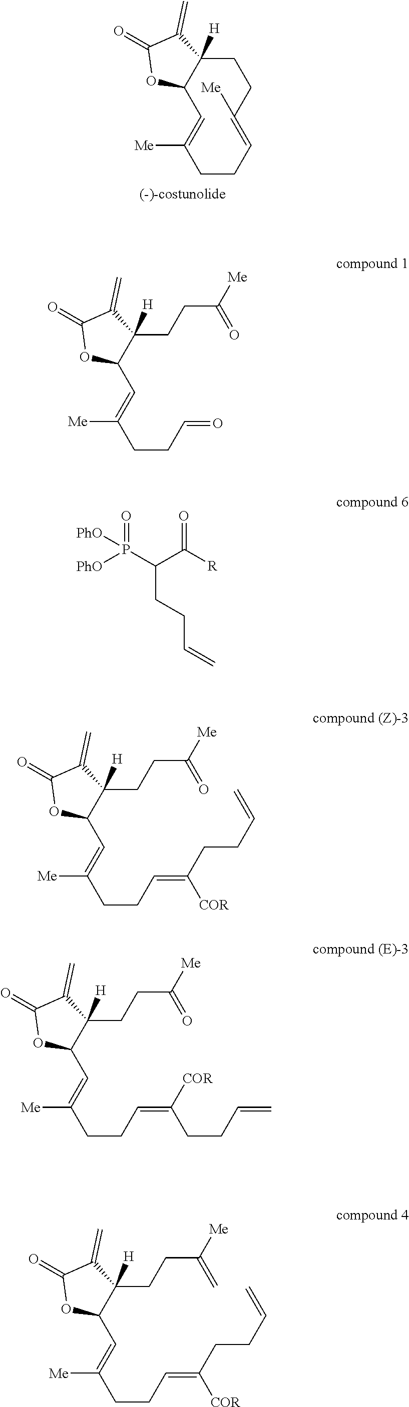 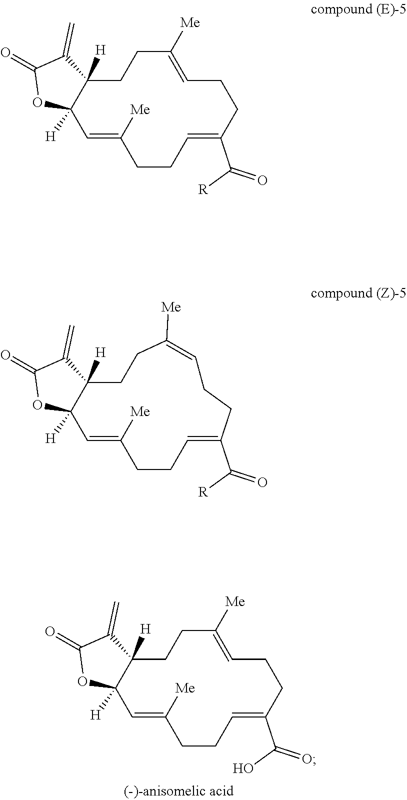
and
wherein the R group in the compound 6, the compound (Z)-3, the compound (E)-3, the compound 4, the compound (E)-5 and the compound (Z)-5 is alkoxy group, aromatic oxygen group, alkylamine group, aromatic amine group, alkyl sulfhydryl group, aromatic sulfhydryl group, or silicon group.

2. The method of claim 1, characterized by, the method of ozonation decomposition in step 1) comprises: introducing ozone into the compound (−)-Costunolide in solution at low temperature until the reaction being finished, adding a reducing reagent for quenching the reaction, removing solvents after raising the reaction system to room temperature, purifying the residue by using silica gel column chromatography to obtain compound 1.

3. The method of claim 1, characterized by, the method of step 2) comprises: adding an alkaline substance into the compound 6 in solution under low temperature, subsequently, adding the compound 1 in solution, then adding a quenching reagent until the reaction being finished, purifying the residue by using silica gel column chromatography to obtain compound (Z)-3 and compound (E)-3.

4. The method of claim 1, characterized by, the method of step 3) comprises: adding the cerium trichloride into a round-bottom bottle, heating under vacuum condition, and stirring for a certain time, filling with inert gas, moving the reaction system into an ice water bath, adding tetrahydrofuran, and then raising the temperature to room temperature and stirring for a certain time; adding a lithium reagent at low temperature, and keeping the same temperature and continuously stirring for a certain time, adding compound (Z)-3 into the reaction system, and stirring for a certain time at the same temperature; quenching the reaction system by adding acetic acid aqueous solution, separating the liquid, and extracting the aqueous phase by ethyl acetate; combining the organic phase, drying, removing solvent, then adding a reagent that promotes elimination to the residue, finally, purifying the residue by silica gel column chromatography to obtain compound 4.

5. The method of claim 1, characterized by, the method of step 4) comprises: adding catalyst of olefin metathesis into the tetraene compound 4 in solution, discharging the residual oxygen from the reaction system under the condition of inert gas atmosphere for a certain time, then raising the temperature of the reaction system until the conversion of tetraene compound 4 being complete, removing solvents, purifying the residue by using silica gel column chromatography to obtain the 14-membered macrocyclic compound (Z)-5 and the 14-membered macrocyclic compound (E)-5.

6. The method of claim 1, characterized by, the method of step 5) comprises: cooling down the 14-membered macrocyclic compound (E)-5 in solution to 0° C., adding a desiliconization reagent dropwise and reacting at the temperature for 1 hour, quenching the reaction system by using saturated ammonium chloride solution, extracting by using ethyl acetate and combining the organic phase after raising the temperature to room temperature; drying and removing solvents, purifying the residue by using silica gel column chromatography to obtain the natural product (−)-Anisomelic Acid.

7. The method of claim 1, characterized by, the method of step 2) further comprises the following step:
2-1) preparing phosphate compound 6 by using the compound 2 and the compound 3 under alkaline condition;
wherein, the chemical formulae of the compound 2 and the compound 3 are shown as: 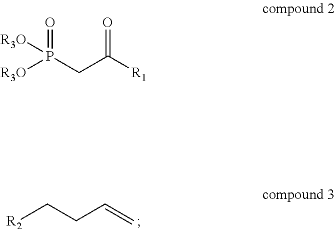
wherein, in compound 2, the R1 group is alkoxy group, aromatic oxygen group, alkylamine group, aromatic amine group, alkyl sulfhydryl group, aromatic sulfhydryl group, or silicon group; R3 group is phenyl group, or trifluoroethyl group, and
wherein, in compound 3, R2 group is chlorine, bromine, iodine, methylsulfonyloxy, p-toluene sulfonyloxy, or trifluoromethylsulfonyloxy.

8. The method of claim 7, characterized by, the method of step 2-1) further comprises the following step: cooling down the compound 2 in solution to 0° C., adding an alkaline substance slowly under an inert gas atmosphere, stirring for a certain time at the temperature, slowly adding the compound 3 in solution in a dropwise manner, and then raising the temperature until the reaction being complete, quenching the reaction system by using saturated ammonium chloride solution, after the temperature being increased to room temperature, extracting by using ethyl acetate, and combining the organic phase; drying, removing solvent, purifying the residue by using silica gel column chromatography to obtain the phosphate compound 6.
